(19) United States
(12) Patent Application Publication
(10) Pub. No.: US 2005/0196370 A1
(43) Pub. Date: Sep. 8, 2005

(54) Stable ophthalmic oil-in-water emulsions with sodium hyaluronate for alleviating dry eye

(76) Inventors: Zhi-Jian Yu, Irvine, CA (US); Stanley W. Huth, Newport Beach, CA (US); Lauren L. Crawford, Mission Viejo, CA (US); James N. Cook, Mission Viejo, CA (US)

Correspondence Address: Advanced Medical Optics, Inc.;Legal Department, 1700 E. St. Andrew Place, Santa Ana, CA, 92705, US

(21) Appl. No.: 11/098,827

(22) Filed: Apr. 4, 2005

Related U.S. Application Data
(63) Continuation-in-part of application No. 10/802,153, filed on Mar. 17, 2004.
Application No. 10/802,153 is a continuation-in-part of application No. 10/392,375, filed on Mar. 18, 2003.

(51) Int. Cl. A61K 7/06; A61K 7/11; A61K 35/78; A61K 9/66

(52) U.S. Cl. 424/70.13; 424/401; 424/70.15; 424/731

(57) ABSTRACT

Stable oil-in-water emulsions are described which contain a demulcent for the treatment of dry eye such as sodium hyaluronate. The oil-in-water emulsions are stable and have anti-microbial activity sufficient for use as contact lens disinfecting solutions.

### RELATED APPLICATIONS

This application is a continuation-in-part of U.S. application Ser. No. 10/802,153, filed Mar. 17, 2004 which is a continuation-in-part of U.S. application Ser. No. 10/392,375, filed Mar. 18, 2003. Both applications are incorporated herein by reference.

### BACKGROUND OF THE INVENTION

1. Field of the Invention

Embodiments of the invention relate to self-emulsifying ophthalmic compositions containing a demulcent, particularly hyaluronic acid, for the treatment and/or relief of dry eye.

2. Description of the Related Art

Dry eye syndrome is a prevalent condition for which there is no cure, although symptoms may be relieved with proper diagnosis and treatment. The condition affects more than 3.2 million American women middle-aged and older alone (Schaumberg D A, Sullivan D A, Buring J E, Dana M R. Prevalence of dry eye syndrome among US women. Am J Ophthalmol 2003 August; 136 (2): 318-26). Contact lens wearers, computer users, patients who live and/or work in dry environments, and patients with autoimmune disease are all particularly susceptible to developing dry eye.

Hyaluronic acid occurs naturally in the human body and has been shown to effectively treat symptoms of dry eye. Though long-term studies have yet to be done, treatment with sodium hyaluronate appears to accelerate recovery of the damaged cornea (Katsuyama I, Arakawa T. A convenient rabbit model of ocular epithelium damage induced by osmotic dehydration. J Ocul Pharmacol Ther 2003 June; 19 (3): 281-9). Sodium hyaluronate eye drops increase precorneal tear film stability and corneal wettability, reduce the tear evaporation rate, and the healing time of corneal epithelium (Aragona P, Di Stefano G, Ferreri F, et al. Sodium hyaluronate eye drops of different osmolarity for the treatment of dry eye in Sjogren's syndrome patients. Br J Ophthalmol 2002 August; 86 (8): 879-84). Sodium hyaluronate can be found in AQuify contact lens comfort drops (CIBA Vision) However, it is difficult to incorporate water soluble polymers such as HA into ophthalmic oil-in-water emulsions.

### SUMMARY OF THE INVENTION

Embodiments of the invention are directed to self-emulsifying composition. Self-emulsifying compositions according to embodiments of the invention generally include oil globules having an average size of less than 1 micron dispersed in an aqueous phase. These globules may include a surfactant component and a polar oil component. In preferred embodiments, the surfactant component and the oil component are selected to self-emulsify when mixed without mechanical homogenization. In preferred embodiments, the self-emulsifying compositions include a first therapeutic component which may include a water-soluble polymer. In preferred embodiments, the surfactant component of the self-emulsifying composition includes one or two surfactants.

In preferred embodiments, that water soluble component of the self-emulsifying composition is hyaluronic acid or salts of hyaluronic acid, polyvinylpyrrolidone (PVP), cellulose polymers, dextran 70, gelatin, polyethylene glycols, polyvinyl alcohol, or povidone. In more preferred embodiments, the cellulose polymer is carboxymethylcellulose or hydroxypropyl methylcellulose. In alternate more preferred embodiments, the polyethylene glycol is PEG 300 or PEG 400.

In preferred embodiments, the oil component of the self-emulsifying composition includes castor oil or a natural oil. In some preferred embodiments, the self-emulsifying composition also includes a chlorite preservative component. In more preferred embodiments, the chlorite preservative component is stabilized chlorine dioxide (SCD), metal chlorites, or mixtures of these preservatives.

In preferred embodiments, the self-emulsifying composition also includes a cationic antimicrobial which is poly[dimethylimino-w-butene-1,4-diyl]chloride, alpha-[4-tris(2-hydroxyethyl)ammonium]-dichloride (Polyquatemium 1®), poly(oxyethyl (dimethyliminio)ethylene dmethyliminio)ethylene dichloride (WSCP®), polyhexamethylene biguanide (PHMB), polyaminopropyl biguanide (PAPB), benzalkonium halides, salts of alexidine, alexidine-free base, salts of chlorhexidine, hexetidine, alkylamines, alkyl di- and tri-amine, tromethamine(2-amino-2-hydroxymethyl-1,3 propanediol), Octenidine (N,N′-(1,10-Decanediyldi-1-(4H)-pyridinyl-4-ylidenebis-[1-octanamine]dihydrochloride, hexamethylene biguanides and their polymers, cetylpyridinium chloride, cetylpyridinium salts, antimicrobial polypeptides, or mixtures of these cationic preservatives.

In preferred embodiments, the surfactant component has a hydrophobic portion which includes a first part oriented proximal to the aqueous phase that is larger than a second part of the hydrophobic portion of the surfactant component oriented towards the interior of the oil globule. More preferably, the surfactant component includes one surfactant with the first part of the hydrophobic portion of the surfactant that contains more atoms than the second part of the hydrophobic portion of the surfactant. In some preferred embodiments, the surfactant component includes two surfactants, a first of said surfactants including a first hydrophobic portion and a second of said surfactants including a second hydrophobic portion, said first hydrophobic portion having a longer chain length than the second hydrophobic portion.

In some embodiments, the self-emulsifying composition also includes an additional surfactant that does not interfere with self-emulsification.

In preferred embodiments, self-emulsifying composition includes a surfactant component which is (a) a compound having at least one ether formed from at least about 1 to 100 ethylene oxide units and at least one fatty alcohol chain having from at least about 12 to 22 carbon atoms; and/or (b) a compound having at least one ester formed from at least about 1 to 100 ethylene oxide units and at least one fatty acid chain having from at least about 12 to 22 carbon atoms; and/or (c) a compound having at least one ether, ester or amide formed from at least about 1 to 100 ethylene oxide units and at least one vitamin or vitamin derivative; and (d) combinations thereof which have no more than two surfactants. In a most preferred embodiment, the surfactant component is Lumulse GRH-40 or TPGS.

In preferred embodiments of the self-emulsifying composition the oil globules have an average size of less than about 0.25 micron. In more preferred embodiments, the oil globules have an average size of less than about 0.15 micron.

In preferred embodiments, the self-emulsifying composition may also include a cationic antimicrobial component having an HLB value significantly higher than an HLB value of the polar oil component.

Embodiments of the invention are directed to therapeutic compositions which include any of the self-emulsifying compositions described above and a second therapeutic component. In preferred embodiments, the second therapeutic component is cyclosporin, prostaglandins, Brimonidine, or Brimonidine salts. In most preferred embodiments, the therapeutic composition includes a surfactant component which is Lumulse-GRH-40 or TPGS.

In preferred embodiments, the self-emulsifying composition may be used as a multipurpose solution for contact lenses.

Embodiments of the invention are directed to methods of treating an eye which includes the steps of administering any of the self-emulsifying compositions described above to an individual in need thereof. Preferably, the treatment is for dry eye. Preferably, the individual is a mammal.

Embodiments of the invention are directed to methods of preparing a self-emulsifying composition which may include the steps of preparing an oil phase which includes a polar oil and a surfactant component, wherein the polar oil and the surfactant component in the oil phase are in the liquid state; preparing an aqueous phase at a temperature that permits self-emulsification; wherein the aqueous phase comprises a water soluble polymer; and mixing the oil phase and the aqueous phase to form an emulsion, without mechanical homogenization. The method may also include forming a paste between the oil phase and a part of the aqueous phase and mixing the paste with the rest of the aqueous phase to form an emulsion.

In preferred embodiments, the water soluble polymer may be selected from hyaluronic acid and salts thereof, polyvinylpyrrolidone (PVP), cellulose polymers, dextran 70, gelatin, polyethylene glycols, polyvinyl alcohol, and povidone. In some preferred embodiments, the cellulose polymer may be carboxymethylcellulose or hydroxypropyl methylcellulose. In some preferred embodiments, the polyethylene glycol may be PEG 300 or PEG 400. In preferred embodiments, the surfactant component includes one or two surfactants.

Further aspects, features and advantages of this invention will become apparent from the detailed description of the preferred embodiments which follow.

### DESCRIPTION OF THE DRAWING

These and other feature of this invention will now be described with reference to the drawings of preferred embodiments which are intended to illustrate and not to limit the invention.

FIGS. 1A and 1B show a flow chart for the preparation of the ophthalmic self-emulsifying compositions described.

### DETAILED DESCRIPTION OF THE PREFERRED EMBODIMENT

Embodiments of the invention are directed to ophthalmic oil-in-water emulsions which contain a water-soluble polymer such as hyaluronic acid. Such emulsions have the advantage of added comfort due to the low amount of surfactant relative to the oil component. This leads to greater comfort for the end user. The integration of emulsions containing therapeutic demulcents into contact lens care compositions, such as multi-purpose, re-wetting and other contact lens care compositions adds the additional utility or benefit of prevention and/or treatment of dry eye and provides lubrication to the lens and/or eye through mechanisms only emulsions can provide. Additional utilities or benefits provided by integrated emulsions in contact lens care compositions may include, without limitation, enhanced contact lens cleaning, prevention of contact lens water loss, inhibition of protein deposition on contact lenses and the like.

There are two problems with incorporation of hyaluronic acid into ophthalmic oil-in-water emulsions. The first is that hyaluronic acid and similar compounds destabilize oil-in-water emulsions and the second problem is maintaining sterility of oil-in-water ophthalmic solutions which contain hyaluronic acid and other demulcents.

An oil-in-water emulsion is usually generated and stabilized by a surfactant emulsifier. Efforts have been made to incorporate ophthalmically acceptable demulcents, such as polyvinylpyrrolidone (PVP), hydroxypropyl methylcellulose (HPMC), or sodium hyaluronate (HA). However, such polymers destabilize oil-in-water emulsions via the Bridging Flocculation effect. The water soluble polymer interacts with the emulsion droplets forming a cluster of emulsion-polymer complexes. When too many emulsion droplets are picked up by the polymer, the overall mass of the cluster becomes so large that the buoyancy overcomes the Brownian motion and creaming occurs.

The second problem is that antimicrobial activity is lost in the presence of a large amount of surfactant containing alkyl chains, such as POE(40) hydrogenated Castor Oil. In fact, Tween 80 is routinely used as a quaternary ammonium neutralizer in antimicrobial activity testing. The surfactant forms micelles, which strongly adsorb the antimicrobial, thereby reducing the activity. Thus, it may be different to maintain antimicrobial activity in the presence of the surfactant(s) which are components of the emulsion.

While hyaluronic acid is clearly a promising agent for the effective treatment of dry eye, a need exists for stable ophthalmic emulsion compositions containing water soluble polymers such as hyaluronic acid. Additionally it is desirable for self-emulsifying compositions capable of treating dry eye to have antimicrobial activity so that the compositions can be maintained free of microbial contamination and for decontamination of contact lenses.

Embodiments of the present invention provide oil-in-water emulsions containing a demulcent which is a water soluble polymer such as hyaluronic acid which are easily prepared and sterilized and which are storage stable as well, as methods of preparing such compositions. These ophthalmic compositions also have a low surfactant to oil ratio for high comfort and employ fewer surfactants to achieve emulsification. Ophthalmic compositions according to the invention are stable and free of microbial growth for at least two years. These compositions employ molecular self-assembly methods to generate macromolecular oil droplet structures at the nanometer scale, and thus represent an example of nanotechnology.

Definitions

The term “emulsion” is used in its customary sense to mean a stable and homogenous mixture of two liquids which do not normally mix such as oil and water.

An “emulsifier” is a substance which aids the formation of an emulsion such as a surfactant. The terms “emulsifier” and “surfactant” are used interchangeably herein. In the context of the present invention, surfactant component means one or more surfactants that are present in the self-emulsifying composition and contribute to the self-emulsification.

The term “stable” is used in its customary sense and means the absence of creaming, flocculation, and phase separation.

The term “demulcent” is used in the usual sense and refers to an agent that relieves irritation of inflamed or abraded lens and/or eye surfaces.

The term “polar oil” means that the oil contains heteroatoms such as oxygen, nitrogen and sulfur in the hydrophobic part of the molecule.

A “multi-purpose composition,” as used herein, is useful for performing at least two functions, such as cleaning, rinsing, disinfecting, rewetting, lubricating, conditioning, soaking, storing and otherwise treating a contact lens, while the contact lens is out of the eye. Such multi-purpose compositions preferably are also useful for re-wetting and cleaning contact lenses while the lenses are in the eye. Products useful for re-wetting and cleaning contact lenses while the lenses are in the eye are often termed re-wetters or “in-the-eye” cleaners.

The term “cleaning” as used herein includes the loosening and/or removal of deposits and other contaminants from a contact lens with or without digital manipulation and with or without an accessory device that agitates the composition.

The term “re-wetting” as used herein refers to the addition of liquid over at least a part, for example, at least a substantial part, of at least the anterior surface of a contact lens.

Therapeutic ophthalmic compositions for the treatment and/or relief of dry eye are disclosed. The ophthalmic compositions include oil-in-water emulsions, preferably self-emulsifying oil-in-water emulsions, along with a therapeutic demulcent and a biocide to control microbial growth. Methods of preparing or making such compositions and methods of using such compositions are also disclosed. The present emulsion-containing compositions are relatively easily and straight forwardly prepared and are storage-stable, for example, having a shelf life at about room temperature of at least about 2 years or more. In addition, the present compositions are advantageously easily sterilized, for example, using sterilizing filtration techniques, and eliminate, or at least substantially reduce, the opportunity or risk for microbial growth if the compositions become contaminated by inclusion of at least one anti-microbial agent.

Preferred embodiments are directed to compositions comprising oil-in-water emulsions for the treatment of dry eye. For this use, one would administer a composition as needed as determined by one skilled in the art. For example, ophthalmic demulcents such as carboxymethylcellulose, other cellulose polymers, dextran 70, gelatin, polyethylene glycols (e.g., PEG 300 and PEG 400), polyvinyl alcohol, povidone and the like and mixtures thereof, may be used in the present ophthalmic compositions, for example, compositions useful for treating dry eye.

In more preferred embodiments, therapeutic demulcents according to the invention include water soluble polymers such as polyvinylpyrrolidone (PVP), hydroxypropyl methylcellulose (HPMC), and hyaluronic acid (HA) and salts thereof.

In most preferred embodiments, the therapeutic agent is hyaluronic acid (HA). HA is a natural polymer. As used herein, the term HA means hyaluronic acid and any of its hyaluronate salts, including, for example, sodium hyaluronate (the sodium salt), potassium hyaluronate, magnesium hyaluronate, and calcium hyaluronate.

HA is a polymer consisting of simple, repeating disaccharide units (glucuronic acid and N-acetyl glycosamine). It is made by connective tissue cells of all animals, and is present in large amounts in such tissues as the vitreous humor of the eye, the synovial fluids of joints, and the roostercomb of chickens. One method of isolating HA is to process tissue such as roostercombs. HA isolated from natural sources can be obtained from commercial suppliers, such as Biomatrix, Anika Therapeutics, ICN, and Pharmacia.

Another method of producing HA is via fermentation of bacteria, such as streptococci. The bacteria are incubated in a sugar rich broth, and excrete HA into the broth. HA is then isolated from the broth and impurities are removed. The molecular weight of HA produced via fermentation may be altered by the sugars placed in the fermentation broth. HA produced via fermentation can be obtained from companies such as Bayer, Genzyme, and Fidia.

In its natural form, HA has a molecular weight in the range of 5×10⁴ up to 1×10⁷ daltons. Its molecular weight may be reduced via a number of cutting processes such as exposure to acid, heat (e.g. autoclave, microwave, dry heat), or ultrasonic waves. HA is soluble in water and can form highly viscous aqueous solutions.

The present compositions preferably include self-emulsifying emulsions. That is, the present oil-in-water emulsions preferably can be formed with reduced amounts of dispersion mixing at shear speed, more preferably with substantially no dispersion mixing at shear speed. Dispersion mixing at shear speed is also known as mechanical homogenization. Mechanical homogenization to form an emulsion typically occurs at shear speeds greater than 1000 r.p.m., more typically at several thousand r.p.m., and even at 10,000 r.p.m. or more. In other words, the present self-emulsifying emulsions preferably can be formed using reduced amounts of shear, and more preferably using substantially no shear. Further, the present emulsions have a relatively low weight ratio of emulsifying component or surfactant component to oil or oily component and, therefore, are advantageously safe and comfortable for topical ophthalmic application. Such oil-in-water emulsions, with a low surfactant to oil ratio, may be more readily prepared via self-emulsification than oil-in-water emulsions with a higher surfactant to oil ratio.

Topical ophthalmic application forms of the present compositions include, without limitation, eye drops for dry eye treatment and for other treatments, forms for the delivery of drugs or therapeutic components into the eye and forms for caring for contact lenses. The present compositions are very useful for treating dry eye and similar conditions, and other eye conditions. In addition, the present compositions are useful in or as carriers or vehicles for drug delivery, for example, a carrier or vehicle for delivery of therapeutic components into or through the eyes.

Contact lens care applications of the present compositions include, without limitation, compositions useful for cleaning, rinsing, disinfecting, storing, soaking, lubricating, re-wetting and otherwise treating contact lenses, including compositions which are effective in performing more than one of such functions, i.e., so called multi-purpose contact lens care compositions, other contact lens care-related compositions and the like. Contact lens care compositions including the present emulsions also include compositions which are administered to the eyes of contact lens wearers, for example, before, during and/or after the wearing of contact lenses.

Embodiments of the invention provide for therapeutic ophthalmic compositions which include oil-in-water emulsions, preferably self-emulsifying oil-in-water emulsions. These oil-in-water emulsions comprise an oil component, for example, and without limitation, castor oil; and an aqueous component which includes two emulsifiers or surfactants or less. The use of only one or two emulsifiers results in a low weight ratio of emulsifying component to oil component and fewer chemical toxicity concerns, resulting in comfort and safety advantages over emulsions employing more than two emulsifiers.

The oily component and the surfactant component or surfactants are advantageously chemically structurally compatible to facilitate self-emulsification of the emulsion. In the context of the present invention, surfactant component means one or two surfactants that are present in the self-emulsifying composition and contribute to the self-emulsification. The one or two surfactants must have an affinity for the selected oil or oils based upon non-covalent bonding interactions between the hydrophobic structures of the surfactant and the oil(s) such that self emulsification can be achieved. In one aspect, affinity relates to the use of a polar oil with a surfactant of similar polarity. As the terms are used herein, a polar oil means that the oil contains heteroatoms such as oxygen, nitrogen and sulfur in the hydrophobic part of the molecule. In a preferred embodiment, the self-emulsifying emulsions described contain at least one polar oil.

In preferred embodiments, the one or two surfactants must be able to form a chemical structure which is wedge or pie section-shaped, with the larger end of the wedge structure closer to the hydrophilic parts of the surfactant structures. That is, the part of the surfactant that is larger is oriented towards the aqueous phase and contains more atoms than the part of the surfactant that is oriented towards the oil phase. When the surfactant component includes two surfactants, the hydrophobic portion of the first surfactant may have a longer chain length than the hydrophobic portion of the second surfactant to promote formation of a wedge shape.

The surfactants useful to form the surfactant component in the present invention advantageously are water-soluble when used alone or as a mixture. These surfactants are preferably non-ionic. The amount of surfactant component present varies over a wide range depending on a number of factors, for example, the other components in the composition and the like. Often the total amount of surfactant component is in the range of about 0.01 to about 10.0 w/w %. It is noted that additional surfactant(s) may be present in the self-emulsifying composition (in addition to the surfactant component) and still fall within the scope of the present invention if the additional surfactant(s) are present at a concentration such that they do not interfere with the self-emulsification.

The ratio, for example, weight ratio, of the surfactant component to the oily component in the present oil-in-water emulsions is selected to provide acceptable emulsion stability and performance, and preferably to provide a self-emulsifying oil-in-water emulsion. Of course, the ratio of surfactant component to oily component varies depending on the specific surfactants and oil or oils employed, on the specific stability and performance properties desired for the final oil-in-water emulsion, on the specific application or use of the final oil-in-water emulsion and the like factors. For example, the weight ratio of the surfactant component to the oily component may range from about 0.01 to 40, preferably from 0.1 to 20, more preferably from 0.2 to 2.0.

Such surfactants function as described herein, provide effective and useful ophthalmic compositions and do not have any substantial or significant detrimental effect on the contact lens being treated by the present compositions, on the wearers of such contact lenses or on the humans or animals to whom such compositions are administered.

One or more oils or oily substances are used to form the present compositions. Any suitable oil or oily substance or combinations of oils or oily substances may be employed provided such oils and/or oily substances are effective in the present compositions, and do not cause any substantial or significant detrimental effect to the human or animal to whom the composition is administered, or to the contact lens being treated, or the wearing of the treated contact lens, or to the wearer of the treated contact lens. The oily component may, for example, and without limitation, be polar in nature and naturally or synthetic derived. Natural oils may be obtained from plants or plant parts such as seeds or they may be obtained from an animal source such as Sperm Whale oil, Cod liver oil and the like. The oil may be a mono, di or triglyceride of fatty acids or mixtures of glycerides, such as Castor oil, Coconut oil, Cod-liver oil, Corn oil, Olive oil, Peanut oil, Safflower oil, Soybean oil and Sunflower oil. The oil may also be comprised of straight chain monoethylene acids and alcohols in the form of esters, such as Jojoba and Sperm Whale oil. The oil may be synthetic, such as silicone oil. The oil also may be comprised of water insoluble non-volatile liquid organic compounds, e.g., a racemic mixture of Vitamin E acetate isomers. Mixtures of the above oil types may also be used.

Oils which are natural, safe, have prior ophthalmic or other pharmaceutical use, have little color, do not easily discolor upon aging, easily form spread films and lubricate surfaces without tackiness are preferred. Castor oil and the like are preferred oils.

In one embodiment, the present invention relates to therapeutic ophthalmic compositions which are self-emulsifying, oil-in-water emulsions which contain a therapeutic demulcent as well as methods of preparing and methods of using such therapeutic ophthalmic compositions. These compositions are useful for treatment and/or relief of dry eye and contact lens care. These emulsions employ molecular self-assembly methods to generate macromolecular oil droplet structures at the nanometer and sub-micron scale and thus represent an example of nanotechnology. The emulsions are easily prepared via molecular self-assembly in milliseconds to minutes. The emulsions can be filter sterilized and are storage-stable. The emulsions employ only one or two surfactant emulsifiers to achieve low surfactant to oil ratios. The compositions are comfortable and non-toxic to the eye.

Topical ophthalmic applications for the emulsions of the present invention include eye drops for dry eye treatment, compositions for delivery of drugs to and via the eye, and contact lens care solutions. Contact lens care solution applications include multipurpose cleaning, rinsing, disinfecting and storage solutions as well as rewetting, in-the-eye cleaning and other solutions for the eye. In preferred embodiments, HA is present at a concentration of at least 0.001% (w/w). Typically, HA is present at a concentration of from 0.01% (w/w) to 0.3% (w/w). Higher concentrations of HA may be preferred if the emulsion concentration is high. Preferably, HA is present at 0.1-0.2% w/w.

The integration of oil-in-water emulsions with water soluble polymer demulcents into eye drops for dry eye treatment, contact lens rewetting and multipurpose solutions adds the additional utility of prevention of dry eye and contact lens water loss by providing an oil layer at the air-tear interface or additionally at the contact lens-tear interface when a contact lens is present. This oil layer acts to prevent dry eye or contact lens water loss by retarding water evaporation and thus loss. The oil layer on the surface of a contact lens can also provide a long-lasting lubrication layer, especially for rigid gas permeable contact lenses. The oil layer on the surface of a contact lens can also inhibit contact lens protein deposition.

The self-emulsifying, oil-in-water emulsions for the therapeutic compositions of the present invention are of two general types. The first type is a one surfactant system. The second type is a two surfactant system. In either case, what is required is that (1) the surfactant(s) must have an affinity for the selected oil or oils based upon non-covalent bonding interactions between the hydrophobic structures of the surfactant and the oil(s) such that self emulsification can be achieved when requirement (2) is simultaneously met; and (2) the surfactant must have a chemical structure which is wedge or pie section-shaped, with the larger end of the wedge structure closer to the hydrophilic part of the surfactant structure. This wedge-shape is believed to induce spherical oil droplet curvature at the aqueous-oil interface due to the molecular self-assembly of adjacent surfactant wedges at the aqueous-oil interface. Thus, the geometry of the wedge-shaped surfactant molecules is intimately related to the oil droplet curvature. Steric repulsion in the aqueous phase between the hydrophilic parts of adjacent surfactant molecules facilitates this. Preferably, these hydrophilic parts consist of polyethyleneoxide chains of an appropriate length. Preferably, the polyethyleneoxide chains are from 7-20 ethyleneoxide units in length. When the aforementioned two structural requirements are met for a surfactant and oil(s) pair(s), an empirical test of self emulsification is conducted while varying the concentrations of the surfactant and oil components. The empirical test of self emulsification is conducted employing the methods of preparing self emulsifying emulsions described herein. An emulsion is considered to be acceptable when it appears to be homogeneous when observed by eye, without any appearance of flocculation, cream or phase separation between the aqueous and oil phase and also when the oil droplet size distribution of the emulsion meets particular product criteria for emulsion stability.

As a practical matter, a surfactant is a good candidate for the self-emulsifying oil-in-water emulsions described herein if the surfactant is able to form droplets of a size range of 0.05 to 1 micron, preferably, 0.05 to 0.25 micron.

Examples of one component surfactant systems include polyethoxylated oils such as PEG castor oils. Polyethoxylated castor oil derivatives are formed by the ethoxylation of castor oil or hydrogenated castor oil with ethylene oxide. Castor oil is generally composed of about 87% ricinoleic acid, 7% oleic acid, 3% linoleic acid, 2% palmitic acid and 1% stearic acid. The reaction of varying molar ratios of ethylene oxide with castor oil yields different chemical products of PEG castor oils. An example of a PEG castor oil is Lumulse GRH-40, produced by Lambent Technologies Corporation (Skokie, Ill.). A preferred example of a single surfactant and oil pair is the surfactant Lumulse GRH-40 and Castor oil.

Lumulse GRH-40 is a 40 mole ethoxylate of hydrogenated Castor oil. Lumulse GRH-40 is produced through the catalytic hydrogenation of Castor oil at the 9-carbon positions of the three ricinoleic acid glycerol ester chains, followed by ethoxylation of the three 12-hydroxy groups of the 12-hydroxystearic acid glycerol esters with about 13 ethoxy groups each. It is believed that self emulsification of Castor oil with Lumulse GRH-40 occurs due to the folding of the 6-carbon alkyl chain distal to the ethoxylated 12-hydroxy group inwards against the remaining 10-carbon alkyl segment of the stearate ester group to form a wedge-shaped hydrophobic part of the molecule, the orientation of the ethoxy groups outwards into the water phase, the orientation of the wedge-shaped hydrophobic part of the molecule into the Castor oil phase (narrow part of the wedge facing inwards away from the aqueous phase) and the affinity of the wedge-shaped hydrophobic part of the molecule for Castor oil.

The optimal amount of Lumulse GRH-40 to use in conjunction with Castor oil is about 0.8 w/w % Lumulse GRH-40 for 1.0 w/w % Castor oil. Higher or lower amounts in conjunction with Castor oil can be used, however, depending upon the desired properties of the final emulsion. In general, the weight ratio of Lumulse GRH-40 to Castor oil is in the range of 0.6 to 0.8, preferably about 0.8.

Lumulse GRH-40 can be combined with other surfactants such as Polysorbate-80 (Tween-80, polyoxyethylene (20) sorbitan mono-oleate) to create self-emulsifying emulsions comprised of two surfactants. In such compositions, self emulsification is believed to be driven principally by the Lumulse GRH-40. The second surfactant (e.g. polysorbate-80) does not interfere with the emulsifying action of the GRH-40 due to the similar chemical structures of the hydrophobic chains of Polysorbate-80 (oleic acid ester chains) and those of Castor oil (12-hydroxyoleic acid ester chains) and Lumulse GRH-40 (stearic acid ester chains). The non-interfering second surfactant is present at low concentration. That is, the concentration of the non-interfering surfactant is low enough such that it does not interfere with the self-emulsification.

Two surfactants may also be selected to match a particular oil or oils with respect to the ability of the surfactants to form a self-emulsifying oil-in-water emulsion for the dry eye treatments according to the invention. Both surfactants must each meet two chemical structural requirements to achieve self emulsification: (1) each surfactant must have an affinity for the selected oil or oils based upon non-covalent bonding interactions between the hydrophobic structures of the surfactant and the oil(s) such that self emulsification can be achieved when requirement (2) is simultaneously met; and (2) the surfactant pair must be able to form a chemical structure which is wedge or pie section-shaped, with the larger end of the wedge structure closer to the hydrophilic parts of the surfactant structures. A preferred example of a surfactant pair which is compatible with an oil is the surfactant raw material Cremophor RH-40, which is comprised of two surfactants, and Castor oil. Cremophor RH-40, from the BASF Corporation in Parsippany N.J., is comprised 75-78% of two surfactants: the trihydroxystearate ester of polyethoxylated glycerol and the hydroxystearate (bis)ester of mixed polyethylene glycols, along with 22-25% free polyethylene glycols. The Cremophor RH-40 raw material thus has two surfactants which are structurally related to each other and to Castor oil. It is believed that the combination of a surfactant with three ester chains with a surfactant with two ester chains, wherein all of the chains have an affinity for each other, allows the formation of a wedge-shaped structure in the presence of Castor oil wherein the two surfactants alternate at the oil droplet interface. Cremophor RH-60, also from BASF, is an example of another surfactant raw material comprised of two surfactants. Cremophor RH-60 is identical to Cremophor RH-40, with the exception that there is a higher derivatization with polyethyleneglycol with RH-60 than with RH-40.

Additional surfactant(s) may be added which may or may not participate in emulsion formation.

Another example of a one component system utilizes a surfactant such as tocopherol polyethyleneglycol-succinate (TPGS, available from Eastman Chemical Company, Kingsport, Tenn.). TPGS can form a wedge with tocopherol in the narrow section, PEG in the outer section and succinate forming a covalent attachment between them.

More generic descriptions of the types of surfactants which can be used in the present invention include surfactants selected from: (a) at least one ether formed from 1 to 100 ethylene oxide units and at least one fatty alcohol chain having from 12 to 22 carbon atoms; (b) at least one ester formed from 1 to 100 ethylene oxide units and at least one fatty acid chain having from 12 to 22 carbon atoms; (c) at least one ether, ester or amide formed from 1 to 100 ethylene oxide units and at least one vitamin or vitamin derivative, and (d) mixtures of the above consisting of no more than two surfactants.

The preparation of the oil-in-water emulsions for the dry eye-treating compositions of the present invention is generally as follows. Non-emulsifying agents which are water soluble components, including the water-soluble polymer demulcent(s), are dissolved in the aqueous (water) phase and oil-soluble components including the emulsifying agents are dissolved in the oil phase. The two phases (oil and water) are separately heated to an appropriate temperature. This temperature is the same in both cases, generally a few degrees to 5 to 10 degrees above the melting point of the highest melting ingredients in the case of a solid or semi-solid oil or emulsifying agent in the oil phase. Where the oil phase is liquid at room temperature, a suitable temperature is determined by routine experimentation with the melting point of the highest melting ingredients in the aqueous phase. In cases where all components of either the oil or water phase are soluble in their respective phase at room temperature, no heating may be necessary. The temperature must be high enough that all components are in the liquid state but not so high as to jeopardize the stability of the components. A working temperature range is generally from about 20° C. to about 70° C. To create an oil-in-water emulsion, the final oil phase is gently mixed into either an intermediate, preferably de-ionized water phase, or the final aqueous phase to create a suitable dispersion and the product is allowed to cool with or without stirring. In the case wherein the final oil phase is first gently mixed into an intermediate water phase, this emulsion concentrate is thereafter mixed in the appropriate ratio with the final aqueous phase. The final aqueous phase includes the water soluble polymer as well as other aqueous-soluble components. In such cases, the emulsion concentrate and the final aqueous phase need not be at the same temperature or heated above room temperature, as the emulsion has already been formed at this point.

Semisolids may form in the process of self-emulsification if the amount of ethylene oxide units in one emulsifier is too large. Generally, if the surfactant or surfactants have more than 10 ethylene oxide units in their structures, the surfactant and oil phase is mixed with a small amount of the total composition water, e.g., about 0.1-10%, to first form a semi-solid substance in the form of a paste, which is thereafter combined with the remaining water. Gentle mixing may then be required until the hydrated emulsifiers are fully dissolved to form the emulsion.

In one embodiment, the surfactant and oil are initially combined and heated. A small amount of the aqueous phase is then added to the oil phase to form a semi-solid substance in the form of a paste. Paste is defined here as a semisolid preparation. The amount of the aqueous phase added may be from 0.1-10%, preferably from 0.5 to 5% and most preferably 1-2%. After the paste is formed, additional water is added to the paste at the same temperature as above. In some embodiments, the amount of water added is 5-20%. The emulsion is then gently mixed. In some embodiments, mixing may occur for 30 minutes to 3 hours.

In a preferred embodiment, the particles are then sized. A Horiba LA-920 particle size analyzer may be used according to the manufacturer's instructions for this purpose. In a preferred embodiment, the particles are between 0.08 and 0.18 microns in size before passing to the next step.

In the next step, the particles may be mixed with other aqueous components such as water, one or more demulcents and buffer (preferably boric acid based). Optionally, electrolytes, such as calcium chloride dihydrate, magnesium chloride hexahydrate, potassium chloride and sodium chloride, and Kollidon 17 NF may be added. While the electrolytes are not necessary to form the emulsions, they are very helpful to preserve ocular tissue integrity by maintaining the electrolyte balance in the eye. Likewise, the buffer is not critical, but a boric acid/sodium borate system is preferred in one embodiment of the invention because a phosphate-based buffer system will precipitate with the preferred electrolytes.

The pH is adjusted to 6.8-8.0, preferably from about 7.3 to 7.7. This pH range is optimal for tissue maintenance and to avoid ocular irritation. A preservative may then be added. In a preferred embodiment, stabilized chlorine dioxide (SCD) (Purogene®) material is added as preservative. (PUROGENE is a trademark of BioCide International, Inc. Norman, Okla., U.S.A., and is also available as Purite® which is a trademark of Allergan, Inc.)

The oil-in-water emulsions of the present invention can be sterilized after preparation using autoclave steam sterilization or can be sterile filtered by any means known in the art. Sterilization employing a sterilization filter can be used when the emulsion droplet (or globule or particle) size and characteristics allows. The droplet size distribution of the emulsion need not be entirely below the particle size cutoff of the sterile filtration membrane to be sterile-filtratable. In cases where the droplet size distribution of the emulsion is above the particle size cutoff of the sterile filtration membrane, the emulsion needs to be able to deform or acceptably change while passing through the filtrating membrane and then reform after passing through. This property is easily determined by routine testing of emulsion droplet size distributions and percent of total oil in the compositions before and after filtration. Alternatively, a loss of a small amount of larger droplet-sized material may be acceptable.

The emulsions of the present invention are generally non-aseptically filtered through a clarification filter before sterile filtration or aseptically clarify-filtered after autoclave steam sterilization. In a preferred embodiment, the emulsion is filter sterilized using a 0.22 micron filter. Preferably, 98-99% of the emulsion should pass through the 0.22 micron filter. Note that particles larger than 0.22 micron may pass through by altering their shape temporarily. In a preferred embodiment, the material is then tested to verify the effectiveness of the sterilization step. Storage is preferably below 25° C. in order to maintain stability. Thereafter, the emulsions are aseptically filled into appropriate containers.

The present invention provides for methods of using ophthalmic compositions, such as the present ophthalmic compositions described elsewhere herein. In one embodiment, the present methods comprise administering a composition of the invention to an eye of a subject, for example, a human or an animal, in an amount and at conditions effective to provide at least one benefit to the eye. In this embodiment, the present composition can employ at least one portion of the composition, for example, a therapeutic component and the like, useful for treating a condition, for example, dry eye and/or one or more other conditions of the eye.

In a very useful embodiment, the present methods comprise contacting a contact lens with a composition of the present invention in an amount and at conditions effective to provide at least one benefit to the contact lens and/or the wearer of the contact lens. In this embodiment, the present composition is employed as at least a portion of a contact lens care composition.

Compositions according to the invention may be used in methods which comprise administering the composition to an eye of a subject, that is a human or animal, in an amount effective in providing a desired therapeutic effect to the subject. Such therapeutic effect may be an ophthalmic therapeutic effect and/or a therapeutic effect directed to one or more other parts of the subject's body or systemically to the subject's body. In preferred embodiments, the therapeutic effect is treatment and/or relief from symptoms of dry eye.

The aqueous phase or component and the oil phase and component used in accordance with the present invention are selected to be effective in the present compositions and to have no substantial or significant deleterious effect, for example, on the compositions, on the use of the compositions, on the contact lens being treated, on the wearer of the treated lens, or on the human or animal in whose eye the present composition is placed.

The liquid aqueous medium or component of the present compositions preferably includes a buffer component which is present in an amount effective to maintain the pH of the medium or aqueous component in the desired range. The present compositions preferably include an effective amount of a tonicity adjusting component to provide the compositions with the desired tonicity.

The aqueous phase or component in the present compositions may have a pH which is compatible with the intended use, and is often in the range of about 4 to about 10. A variety of conventional buffers may be employed, such as phosphate, borate, citrate, acetate, histidine, tris, bis-tris and the like and mixtures thereof. Borate buffers include boric acid and its salts, such as sodium or potassium borate. Potassium tetraborate or potassium metaborate, which produce boric acid or a salt of boric acid in solution, may also be employed. Hydrated salts such as sodium borate decahydrate can also be used. Phosphate buffers include phosphoric acid and its salts; for example, M₂HPO₄ and MH₂PO₄, wherein M is an alkali metal such as sodium and potassium. Hydrated salts can also be used. In one embodiment of the present invention, Na₂HPO₄. 7H₂O and NaH₂PO₄.H₂O are used as buffers. The term phosphate also includes compounds that produce phosphoric acid or a salt of phosphoric acid in solution. Additionally, organic counter-ions for the above buffers may also be employed. The concentration of buffer generally varies from about 0.01 to 2.5 w/v % and more preferably varies from about 0.05 to about 0.5 w/v %.

The type and amount of buffer are selected so that the formulation meets the functional performance criteria of the composition, such as surfactant and shelf life stability, antimicrobial efficacy, buffer capacity and the like factors. The buffer is also selected to provide a pH, which is compatible with the eye and any contact lenses with which the composition is intended for use. Generally, a pH close to that of human tears, such as a pH of about 7.45, is very useful, although a wider pH range from about 6 to about 9, more preferably about 6.5 to about 8.5 and still more preferably about 6.8 to about 8.0 is also acceptable. In one embodiment, the present composition has a pH of about 7.0.

The osmolality of the present compositions may be adjusted with tonicity agents to a value which is compatible with the intended use of the compositions. For example, the osmolality of the composition may be adjusted to approximate the osmotic pressure of normal tear fluid, which is equivalent to about 0.9 w/v % of sodium chloride in water. Examples of suitable tonicity adjusting agents include, without limitation, sodium, potassium, calcium and magnesium chloride; dextrose; glycerin; propylene glycol; mannitol; sorbitol and the like and mixtures thereof. In one embodiment, a combination of sodium chloride and potassium chloride are used to adjust the tonicity of the composition.

Tonicity agents are typically used in amounts ranging from about 0.001 to 2.5 w/v %. These amounts have been found to be useful in providing sufficient tonicity for maintaining ocular tissue integrity. Preferably, the tonicity agent(s) will be employed in an amount to provide a final osmotic value of 150 to 450 mOsm/kg, more preferably between about 250 to about 330 mOsm/kg and most preferably between about 270 to about 310 mOsm/kg. The aqueous component of the present compositions more preferably is substantially isotonic or hypotonic (for example, slightly hypotonic, e.g., about 240 mOsm/kg) and/or is ophthalmically acceptable. In one embodiment, the compositions contain about 0.14 w/v % potassium chloride and 0.006 w/v % each of calcium and/or magnesium chloride.

In addition to tonicity and buffer components, the present compositions may include one or more other materials, for example, as described elsewhere herein, in amounts effective for the desired purpose, for example, to treat contact lenses and/or ocular tissues, for example, to provide a beneficial property or properties to contact lenses and/or ocular tissues, contacted with such compositions.

In one embodiment, the compositions include a second therapeutic agent in addition to the water-soluble polymer for treatment of dry eye. The compositions of the present invention are useful, for example, as a carrier or vehicle, for the delivery of at least one additional therapeutic agent to or through the eye. Any suitable therapeutic component may be included in the present compositions provided that such therapeutic component is compatible with the remainder of the composition, does not unduly interfere with the functioning and properties of the remainder of the composition, is effective, for example, to provide a desired therapeutic effect, when delivered in the present composition and is effective when administered to or through the eye. For example, in a very useful embodiment, the delivery of hydrophobic therapeutic components or drugs to or through the eye may be accomplished. Without wishing to limit the invention to any particular theory or mechanism of operation, it is believed that the oily component and the hydrophobic constituents of the surfactant components facilitate hydrophobic therapeutic components remaining soluble, stable and effective in the present compositions.

According to this aspect of the invention, an effective amount of a desired second therapeutic agent or component preferably is physically combined or mixed with the other components of a composition of the present invention to form a therapeutic component-containing composition within the scope of the present invention.

While compositions for the delivery of therapeutic agents to or through the eye are a preferred embodiment, the self-emulsifying compositions described herein can be use for delivery of therapeutics through other means including, but not limited to oral, rectal, vaginal, parenteral, intramuscular, intraperitoneal, intraarterial, intrathecal, intrabronchial, subcutaneous, intradermal, intravenous, nasal, buccal and sublingual.

The type of second therapeutic agent or agents used will depend primarily on the therapeutic effect desired, for example, the disease or disorder or condition to be treated. These therapeutic agents or components include a broad array of drugs or substances currently, or prospectively, delivered to or through the eye in topical fashion or otherwise. Examples of useful additional therapeutic components which may be used in conjunction with a treatment for dry eye include, but not limited to:
(1) anti-infective and anti-microbial substances including quinolones, such as ofloxacin, ciprofloxacin, norfloxacin, gatifloxacin and the like; beta-lactam antibiotics, such as cefoxitin, n-formamidoyl-thienamycin, other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin sulfonamides and the like; aminoglycoside antibiotics, such as gentamycin, kanamycin, amikacin, sisomicin, tobramycin and the like; naladixic acid and analogs thereof and the like; antimicrobial combinations, such as fluealanine/pentizidone and the like; nitrofurazones; and the like and mixtures thereof;
(2) anti-allergy agents, antihistaminics, anti-hypertensive agents and decongestants, such as pyrilamine, chlorpheniramine, phenylephrine hydrochloride, tetrahydrazoline hydrochloride, naphazoline hydrochloride, oxymetazoline, antazoline, and the like and mixtures thereof;
(3) anti-inflammatories, such as cortisone, hydrocortisone, hydrocortisone acetate, betamethansone, dexamethasone, dexamethasone sodium phosphate, prednisone, methylprednisolone, medrysone, fluorometholone, fluocortolone, prednisolone, prednisolone sodium phosphate, triamcinolone, sulindac, salts and corresponding sulfides thereof, and the like and mixtures thereof;
(4) non-steroid anti-inflammatory drug (NSAID) components, such as those which do or do not include a carboxylic (—COOH) group or moiety, or a carboxylic derived group or moiety; NSAID components which inhibit, either selectively or non-selectively, the cyclo-oxygenase enzyme, which has two (2) isoforms, referred to as COX-1 and COX-2; phenylalkoanoic acids, such as diclofenac, flurbiprofen, ketorolac, piroximcam, suprofen and the like; indoles such as indomethacin and the like; diarylpyrazoles, such as celecoxib and the like; pyrrolo pyrroles; and other agents that inhibit prostaglandin synthesis and the like and mixtures thereof;
(5) miotics and anticholinergics, such as echothiophate, pilocarpine, physostigmine salicylate, diisopropylfluorophosphate, epinephrine, dipivolyl epinephrine, neostigmine, echothiopate iodide, demecarium bromide, carbachol, methacholine, bethanechol, and the like and mixtures thereof;
(6) mydriatics, such as atropine, homatropine, scopolamine, hydroxyamphetamine, ephedrine, cocaine, tropicamide, phenylephrine, cyclopentolate, oxyphenonium, eucatropine, and the like and mixtures thereof;
(7) antiglaucoma drugs, for example, prostaglandins, such as those described in U.S. Pat. Nos. 6,395,787 and 6,294,563, which are herein incorporated by reference in their entirety, adrenergic agonists such as quinoxalines and quinoxaline derivatives, such as (2-imidozolin-2-ylamino)quinoxaline, 5-halide-6-(2-imidozolin-2-ylamino)quinoxaline, for example, 5-bromo-6-(2-imidozolin-2-ylamino)quinoxaline and brimonidine and its derivatives, such as those described in U.S. Pat. No. 6,294,563, which is herein incorporated by reference in its entirety and the like, timolol, especially as the maleate salt and R-timolol and timolol derivatives and a combination of timolol or R-timolol with pilocarpine and the like; epinephrine and epinephrine complex or prodrugs such as the bitartrate, borate, hydrochloride and dipivefrin derivatives and the like; hyperosmotic and dipivefrin derivatives and the like; betaxolol, hyperosmotic agents, such as glycerol, mannitol and urea and the like and mixtures thereof;
(8) antiparasitic compounds and/or anti-protozoal compounds, such as ivermectin; pyrimethamine, trisulfapyrimidine, clindamycin and corticosteroid preparations and the like and mixtures thereof;
(9) antiviral compounds, such as acyclovir, 5-iodo-2′-deoxyuridine (IDU), adenosine arabinoside (Ara-A), trifluorothymidine, interferon and interferon inducing agents, such as Poly I:C and the like and mixtures thereof;
(10) carbonic anhydrase inhibitors, such as acetazolamide, dichlorphenamide, 2-(p-hydroxyphenyl)thio-5-thiophenesulfonamide, 6-hydroxy-2-benzothiazole-sulfonamide 6-pivaloyloxy-2-benzothiazolesulfonamide and the like and mixtures thereof;
(11) anti-fungal agents, such as amphotericin B, nystatin, flucytosine, natamycin, and miconazole and the like and mixtures thereof;
(12) pain-relieving and anesthetic agents, such as etidocaine, cocaine, benoxinate, dibucaine dydrochloride, dyclonine hydrocholoride, naepaine, phenacaine hydrochloride, piperocaine, proparacaine hydrochloride, tetracaine hydrochloide, hexylcaine, bupivacaine, lidocaine, mepivacaine and prilocaine and the like and mixtures thereof;
(13) ophthalmic diagnostic agents, such as
(a) those used to examine the retina, such as choride-sodium fluorescein and the like and mixtures thereof;
(b) those used to examine the conjunctiva, cornea and lacrimal structures, such as fluorescein and rose Bengal and the like and mixtures thereof; and
(c) those used to examine abnormal pupillary responses such as methacholine, cocaine, adrenaline, atropine, hydroxyamphetamine and pilocarpine and the like and mixtures thereof;
(14) ophthalmic agents used as adjuncts in surgery, such as alpha-chymotrypsin, and hyaluronidase and the like; visco-elastic agents, such as hyaluronates and the like and mixtures thereof;
(15) chelating agents, such as ethylenediamine tetraacetate (EDTA) and deferoxamine and the like; and mixtures thereof;
(16) immunosuppressive agents and anti-metabolites, such as methotrexate, cyclophosphamide, 6-mercaptopurine, cyclosporins such A through I and azathioprine and the like; and mixtures thereof;
(17) angiostatic agents;
(18) muco-secretogogue agents;
(19) proteins and growth factors such as epidermal growth factor;
(20) vitamins and vitamin derivatives such as vitamins A, B12, C, D, E, folic acid and their derivatives;
(21) combinations of the above such as antibiotic/anti-inflammatory as in neomycin sulfate-dexamethasone sodium phosphate, quinolone-NSAID and the like; and concomitant anti-glaucoma therapy, such as timolol maleate-aceclidine and the like.

When a second therapeutic component is present in the compositions of the present invention, the amount of such therapeutic component in the composition preferably is effective to provide the desired therapeutic effect to the human or animal to whom the composition is administered.

Typically, when a second therapeutic component is present, the compositions comprising oil-in-water emulsions of the present invention may contain from or at least about 0.001%, for example, about 0.01%, to about 5% (w/v) of the therapeutic component, e.g., medicament or pharmaceutical, on a weight to weight basis. Thus, for example, from one drop of liquid composition which contains about 25 mg of composition, one would obtain about 0.0025 mg to about 1.25 mg of therapeutic component.

The particular therapeutic component, e.g., drug or medicament, used in the pharmaceutical compositions of this invention is the type which a patient would require or benefit from for the treatment, e.g., pharmacological treatment, of a condition which the patient has or is to be protected from or from which the patient is suffering. For example, if the patient is suffering from glaucoma, the drug of choice may be timolol and/or one or more other anti-glaucoma components.

It is within the knowledge of one skilled in the art to determine the correct amounts of therapeutic component, e.g., drug, to be added to a composition of the invention in order to assure the efficacious delivery of the desired therapeutic component.

In another embodiment, the present compositions are useful as multi-purpose care compositions, rigid gas permeable soaking and conditioning solutions, rewetting compositions and cleaning compositions, for example, in-the-eye cleaners, for contact lens care.

All types of contact lenses may be cared for using compositions of the present invention. For example, the contact lenses may be soft, rigid and soft or flexible gas permeable, silicone hydrogel, silicon non-hydrogel and conventional hard contact lenses.

A multi-purpose composition, as used herein, is useful for performing at least two functions, such as cleaning, rinsing, disinfecting, rewetting, lubricating, conditioning, soaking, storing and otherwise treating a contact lens, while the contact lens is out of the eye. Such multi-purpose compositions preferably are also useful for re-wetting and cleaning contact lenses while the lenses are in the eye. Products useful for re-wetting and cleaning contact lenses while the lenses are in the eye are often termed re-wetters or “in-the-eye” cleaners. The term “cleaning” as used herein includes the loosening and/or removal of deposits and other contaminants from a contact lens with or without digital manipulation and with or without an accessory device that agitates the composition. The term “re-wetting” as used herein refers to the addition of water over at least a part, for example, at least a substantial part, of at least the anterior surface of a contact lens.

Although the present compositions are very effective as multi-purpose contact lens care compositions, the present compositions, with suitable chemical make-ups, can be formulated to provide a single contact lens treatment. Such single treatment contact lens care compositions, as well as the multi-purpose contact lens care compositions are included within the scope of the present invention.

Methods for treating a contact lens using the herein described compositions are included within the scope of the invention. In general, such methods comprise contacting a contact lens with such a composition at conditions effective to provide the desired treatment to the contact lens.

The contact lens can be contacted with the composition, often in the form of a liquid aqueous medium, by immersing the lens in the composition. During at least a portion of the contacting, the composition containing the contact lens can be agitated, for example, by shaking the container containing the composition and contact lens, to at least facilitate the contact lens treatment, for example, the removal of deposit material from the lens. Before or after such contacting step, in contact lens cleaning, the contact lens may be manually rubbed to remove further deposit material from the lens. The cleaning method may optionally also include rinsing the lens prior to or after the contacting step and/or rinsing the lens substantially free of the composition prior to returning the lens to the wearer's eye.

In addition, methods of applying or administering artificial tears, washing eyes and irrigating ocular tissue, for example, before, during and/or after surgical procedures, are included within the scope of the present invention. The present compositions, as described elsewhere herein, are useful as artificial tears, eyewash and irrigating compositions which can be used, for example, to replenish/supplement natural tear film, to wash, bathe, flush or rinse the eye following exposure to a foreign entity, such as a chemical material or a foreign body or entity, or to irrigate ocular tissue subject to a surgical procedure. Foreign entities in this context include, without limitation, one or more of pollen, dust, ragweed and other foreign antigens, which cause adverse reactions, such as allergic reactions, redness, itching, burning, irritation, and the like in the eye.

The present compositions, having suitable chemical make-ups, are useful in each of these, and other, in-the-eye applications. These compositions can be used in in-the-eye applications in conventional and well-known manners. In other words, a composition in accordance with the present invention can be used in an in-the-eye application in a substantially similar way as a conventional composition is used in a similar application. One or more of the benefits of the present compositions, as discussed elsewhere herein, are provided as the result of such in-the-eye use.

A cleaning component may be included in the present compositions useful to clean contact lenses. When present, the cleaning component should be present in an amount effective to at least facilitate removing, and preferably effective to remove, debris or deposit material from a contact lens.

In one embodiment, cleaning surfactants are employed. A cleaning component can be provided in an amount effective to at least facilitate removing deposit material from the contact lens. Types of deposit material or debris which may be deposited on the lens include proteins, lipids, and carbohydrate-based or mucin-based debris. One or more types of debris may be present on a given lens.

The cleaning surfactant component employed may be selected from surfactants conventionally employed in the surfactant cleaning of contact lenses. Among the preferred surfactants are non-ionic surfactants such Pluronic and Tetronic series surfactants, both of which are block copolymers of propylene oxide and ethylene oxide, available from BASF Corp. Performance Chemicals, Mount Olive, N.J., and the like, for example, one or more vitamin derivative components, for example, vitamin E TPGS (D-alpha-tocopheryl polyethylene glycol 1000 succinate).

In one embodiment, a composition in accordance with the present invention containing such a cleaning surfactant component has a surfactant concentration of between about 0.01 and 1.00 w/v %. However, higher or lower amounts may be used.

The present compositions may further comprise one or more antimicrobial agents (i.e., preservatives or disinfectants) to preserve the compositions from microbial contamination and/or disinfect contact lenses. The amount of the disinfectant component present in the liquid aqueous medium is effective to disinfect a contact lens placed in contact with the composition.

In one embodiment, for example, when a multi-purpose contact lens composition is desired, the disinfectant component includes, but is not limited to, quaternary ammonium salts used in ophthalmic applications such as poly[dimethylimino-w-butene-1,4-diyl]chloride, alpha-[4-tris(2-hydroxyethyl)ammonium]-dichloride (chemical registry number 75345-27-6, available under the trademark Polyquaternium 1® (from Onyx Corporation), poly(oxyethyl(dimethyliminio)ethylene dmethyliminio)ethylene dichloride sold under the trademark WSCP by Buckman laboratories, Inc. in Memphis, Tenn., benzalkonium halides, salts of alexidine, alexidine-free base, salts of chlorhexidine, hexetidine, alkylamines, alkyl di- and tri-amine, tromethamine(2-amino-2-hydroxymethyl-1,3propanediol), hexamethylene biguanides and their polymers, cetylpyridinium chloride, cetylpyridinium salts, antimicrobial polypeptides, and the like and mixtures thereof. A particularly useful disinfectant component is selected from one or more (mixtures) of polyhexamethylene biguanide (PHMB), Polyquaternium-1, ophthalmically acceptable salts thereof, and the like and mixtures thereof.

Disinfectant component selection for the oil-in-water emulsions according to embodiments of the invention can be facilitated by using the HLB (Hydrophile-Lipophile Balance) system. The HLB number of the oil component can be obtained from the supplier or from compiled lists in the literature. The HLB number for simple alcohol ethoxylate surfactants may be readily calculated. HLB values for other ethoxylates may be determined experimentally. Overall chemical structure (e.g., branched, linear, aromatic) is also a variable. HLB values are additive; therefore, if two different surfactants or oils are present, the HLB will be the weighted average of the HLB values for each component. In preferred embodiments of the invention, the HLB for the cationic antimicrobial component is significantly higher than the HLB of the oil component. More preferably, the cationic antimicrobial has an HLB value at least 2 HLB units higher than the HLB value of the oil component. Yet more preferably, the cationic antimicrobial has an HLB value at least 5 HLB units higher than the HLB value of the oil component.

The salts of alexidine and chlorhexidine can be either organic or inorganic and are typically disinfecting gluconates, nitrates, acetates, phosphates, sulphates, halides and the like. Generally, the hexamethylene biguanide polymers, also referred to as polyaminopropyl biguanide (PAPB), have molecular weights of up to about 100,000. Such compounds are known and are disclosed in U.S. Pat. No. 4,758,595 which is incorporated in its entirety by reference herein.

The disinfectant components useful in the present invention are preferably present in the present compositions in concentrations in the range of about 0.00001% to about 2% (w/v).

More preferably, the disinfectant component is present in the present compositions at an ophthalmically acceptable or safe concentration such that the user can remove the disinfected lens from the composition and thereafter directly place the lens in the eye for safe and comfortable wear.

When a contact lens is desired to be disinfected by a disinfectant component, an amount of disinfectant effective to disinfect the lens is used. Preferably, such an effective amount of the disinfectant reduces the microbial burden on the contact lens by one log order, in three hours. More preferably, an effective amount of the disinfectant reduces the microbial load by one log order in one hour.

The disinfectant component is preferably provided in the present composition, and is more preferably soluble in the aqueous component of the present composition.

The present compositions may include an effective amount of a preservative component. Any suitable preservative or combination of preservatives may be employed. Examples of suitable preservatives include, without limitation, Purogene®, polyhexamethylene biguanide (PHMB), Polyquaternium-1, ophthalmically acceptable salts thereof, and the like and mixtures thereof, benzalkonium chloride, methyl and ethyl parabens, hexetidine and the like and mixtures thereof. The amount of preservative components included in the present compositions are such to be effective in preserving the compositions and can vary based on the specific preservative component employed, the specific composition involved, the specific application involved, and the like factors. Preservative concentrations often are in the range of about 0.00001% to about 0.05% or about 0.1% (w/v) of the composition, although other concentrations of certain preservatives may be employed.

Very useful examples of preservative components in the present invention include, but are not limited to, chlorite components. Specific examples of chlorite components useful as preservatives in accordance with the present invention include stabilized chlorine dioxide (SCD), metal chlorites, and the like and mixtures thereof. Technical grade (or USP grade) sodium chlorite is a very useful preservative component. The exact chemical composition of many chlorite components, for example, SCD, is not completely understood. The manufacture or production of certain chlorite components is described in McNicholas U.S. Pat. No. 3,278,447, which is incorporated in its entirety by reference herein. Specific examples of useful SCD products include that sold under the trademark Dura Klor by Rio Linda Chemical Company, Inc., that sold under the trademark Anthium Dioxide® by International Dioxide, Inc. North Kingstown, R.I., that sold under the trademark Carnebon 200 by International Dioxide, Inc., OcuPure® by Advanced Medical Optics, Inc., Santa Ana, Calif., and Purogene® by BioCide International, Norman, Okla. (also known as Purite®, available from Allergan, Inc.).

Other useful preservatives include antimicrobial peptides. Among the antimicrobial peptides which may be employed include, without limitation, defensins, peptides related to defensins, cecropins, peptides related to cecropins, magainins and peptides related to magainins and other amino acid polymers with antibacterial, antifungal and/or antiviral activities. Mixtures of antimicrobial peptides or mixtures of antimicrobial peptides with other preservatives are also included within the scope of the present invention.

The compositions of the present invention may include viscosity modifying agents or components, such as cellulose polymers, including hydroxypropyl methyl cellulose (HPMC), hydroxyethyl cellulose (HEC), ethyl hydroxyethyl cellulose, hydroxypropyl cellulose, methyl cellulose and carboxymethyl cellulose; carbomers (e.g. carbopol. RTM); polyvinyl alcohol; polyvinyl pyrrolidone; alginates; carrageenans; and guar, karaya, agarose, locust bean, tragacanth and xanthan gums. Such viscosity modifying components are employed, if at all, in an amount effective to provide a desired viscosity to the present compositions. The concentration of such viscosity modifiers will typically vary between about 0.01 to about 5% w/v of the total composition, although other concentrations of certain viscosity modifying components may be employed.

It is desirable in some instances to include sequestering agents or components in the present compositions in order to, and in an amount effective to, bind metal ions, which, for example, might otherwise stabilize cell membranes of microorganisms and thus interfere with optimal disinfection activity. Alternatively, it is desirable in some instances to bind metal ions to prevent their interaction with other species in the compositions. Sequestering agents are included, if at all, in amounts effective to bind at least a portion, for example, at least a major portion of the metal ions present. Such sequestering components usually are present in amounts ranging from about 0.01 to about 0.2 w/v %. Examples of useful sequestering components include, without limitation ethylene-diaminetetraacetic acid (EDTA) and its potassium or sodium salts and low molecular weight organic acids such as citric and tartaric acids and their salts, e.g., sodium salts.

The present compositions may comprise effective amounts of one or more additional components. For example, one or more conditioning components or one or more contact lens wetting agents and the like and mixtures thereof may be included. Acceptable or effective concentrations for these and other additional components in the compositions of the invention are readily apparent to the skilled practitioner.

Each of the components may be present in either a solid or liquid form of the present compositions. When the additional component or components are present as a solid, they can either be intimately admixed such as in a powder or compressed tablet or they can be substantially separated, although in the same particles, as in an encapsulated pellet or tablet. The additional component or components can be in solid form until desired to be used, whereupon they can be dissolved or dispersed in the aqueous component of the present composition in order to, for example, effectively contact the surface of a contact lens.

When any component is included, it is preferably compatible under typical use and storage conditions with the other components of the composition.

In certain embodiments, an antimicrobial activity of the ophthalmic compositions described herein increases after production. Post-production treatment may include storage of the composition for a period of time from one week to several months, preferably two to six weeks, and most preferably, at least about one month post production. The increase in microbial activity may also be enhanced by treatment with heat, pressure or oxidizing conditions. A combination of treatments may be used. For example, the composition may be stored at a temperature of 30-50° C., more preferably, about 40° C. for a period of at least about two weeks, most preferably, one month.

The ophthalmic compositions according to the invention have the following unexpected properties:
1) When the demulcent (HA, PVP, etc.) concentration is reduced to a certain level, the oil-in-water emulsion is stabilized for a duration of at least 2 years. Phase separation occurs depending upon the demulcent concentration. Instability and creaming may occur within a few days.
2) When hyaluronic acid concentration is high and the emulsion concentration is low, then the HA/emulsion solution is unstable. As the HA concentration decreases and the emulsion concentration increases, the HA/emulsion solution is stabilized. In other words, at a certain HA to emulsion ratio, the solution changes from unstable to stable.
3) It was unexpectedly discovered that certain polymeric quaternary amines such as PHMB and WSCP are not antimicrobially inactivated in combination with the HA containing emulsion formulations.
4) It was also discovered that such an HA/Emulsion combination can better alleviate contact lens wear dryness by providing lubrication and by retarding tear evaporation.

It will be understood by those of skill in the art that numerous and various modifications can be made without departing from the spirit of the present invention. Therefore, it should be clearly understood that the following examples are illustrative only and are not intended to limit the scope of the present invention.

### EXAMPLES

### Example 1

Method of Preparing Ophthalmic Solution

Detailed methods of preparing self-emulsifying compositions may be found in U.S. application Ser. No. 10/802,153, filed Mar. 17, 2004 which is incorporated herein by reference. The following example describes a one-component surfactant system. In this example, PEG-40 hydrogenated castor oil, a 40 mole ethoxylated derivative of hydrogenated castor oil, is exemplified. Reference is made to FIG. 1 and Table 1. FIG. 1 shows a flow chart for the method. Table 1 shows amounts of the various components for this example.

PEG-40 hydrogenated castor oil (Lumulse GRH-40, Lambent Technologies Corp., Skokie, Ill.) and castor oil were heated. The temperature must be high enough that all components are in the liquid state but not so high as to jeopardize the stability of the components. In the present example, a temperature of 60+/−2° C. was used.

A small amount of the total water (1%) was added at 60+/−2° C., to form a transparent white paste. The paste was mixed until the mixture was homogenous. After the paste was formed, more water was added to the paste between 50-62° C. In this example, 7% of the total water was added and mixing was carried out for 1 hour at 200-1000 rpm until the mixture was homogeneous. At this stage, an emulsion concentrate had formed.

The particles (droplets) were then sized using a Horiba LA-920 particle size analyzer according to the manufacturer's instructions. Particles which were between 0.08 and 0.18 microns in size were allowed to pass to the next step.

The emulsion concentrate was mixed with a separately prepared solution of the remaining water, buffer, electrolytes (calcium chloride dihydrate, magnesium chloride hexahydrate, potassium chloride and sodium chloride) and Kollidon 17 NF (polyvinyl pyrrolidone or povidone) (BASF Corporation, Parsippany N.J.) (see Table 1) for about 30 minutes. While the electrolytes are not necessary to form the emulsions, they are very helpful to preserve ocular tissue integrity by maintaining the electrolyte balance in the eye. Likewise, the buffer is not critical to form the emulsion, but is necessary to properly maintain a compatible ocular pH. A boric acid/sodium borate buffer system is preferred because a phosphate-based buffer system will precipitate with the electrolytes. Water soluble polymers such as demulcents for the treatment of dry eye may be added at this stage to form other embodiments of the present invention.

The pH was adjusted to 7.35 to 7.55 with ION NaOH. This pH range is optimal for tissue maintenance and to avoid ocular irritation and is the optimal pH range for stability of Purogene® which was added as a preservative. Sodium Chlorite was added according to the calculation shown in Table 1. Thereafter, pH was checked and adjusted to pH 7.5+/−0.2 with ION NaOH. Note that the pH may only be adjusted with a base such as 10 N NaOH after the addition of Purogene®, as high local solution concentrations of acid formed during acid pH adjustment will cause destruction of the Purogene®.

In the next step, the emulsion was stored covered in the dark at less than 25° C. until sterile filtered. Maximum storage time is 72 hours.

The composition was then filter sterilized using a 0.22 micron filter. 98-99% of the emulsion passed through the 0.22 micron filter. Note that particles larger than 0.22 micron may pass through by altering their shape temporarily. The material was then tested to verify the effectiveness of the sterilization step. The material was then bottled and stored. Pre-fill release specifications for this example were pH 7.3-7.7, mean particle size of 0.09-0.17 microns and physical appearance of a milky white solution. Post-fill release specifications were pH 7.3-7.7, potential chlorine dioxide of 60-70 ppm, castor oil 1.1-1.4% (w/w), Kollidon 17 NF 0.2-0.4% (w/w), osmolality 250-280 mOsm/kg, and sterility USP.

TABLE 1
EMULSION FORMULATION FOR EXAMPLE 1

| Ingredient/Component | Amount/1000 g |
|---|---|
| Lumulse GRH-40 | 10 |
| Castor oil | 12.5 |
| Boric Acid | 6.0 |
| Sodium Borate | 0.35 |
| Calcium Chloride dihydrate | 0.06 |
| Magnesium Chloride hexahydrate | 0.06 |
| Potassium Chloride | 1.4 |
| Sodium Chloride | 3.5 |
| Kollidon 17 PF | 3.0 |
| 10 N Sodium Hydroxide | pH adjust |
| Sodium chlorite | 0.087 |
| Purified Water, USP | QC |
| Sterile filter, 0.22 micron | |

### Example 2

### Characterization of Emulsions Containing HA

Empirical data has shown that hyaluronic acid in certain concentrations can destabilize the emulsion, so as to cause creaming. Examples 2 and 3 illustrate stable and unstable combinations (designation of “unstable” indicates that creaming was observed) with the emulsion formulation and sodium hyaluronate. The formulations in the following examples were prepared essentially as described in Example 1.

TABLE 2
Emulsion formulations for Example 2.

| Ingredients | % w/w | % w/w | % w/w | % w/w |
|---|---|---|---|---|
| Sodium hyaluronate | 0.1 | 0.2 | 0.3 | 0.4 |
| Castor Oil | 1.25 | 1.25 | 1.25 | 1.25 |
| POE(40) Hydrogenated Castor | 1 | 1 | 1 | 1 |
| Oil | | | | |
| Sodium Chlorite | 65 ppm | 65 ppm | 65 ppm | 65 ppm |
| WSCP | 3 ppm | 3 ppm | 3 ppm | 3 ppm |
| Boric Acid | 0.6 | 0.6 | 0.6 | 0.6 |
| Sodium Borate Decahydrate | 0.035 | 0.035 | 0.035 | 0.035 |
| Calcium chloride dihydrate | 0.006 | 0.006 | 0.006 | 0.006 |
| Magnesium chloride | 0.006 | 0.006 | 0.006 | 0.006 |
| hexahydrate | | | | |
| Potassium chloride | 0.14 | 0.14 | 0.14 | 0.14 |
| Sodium chloride | 0.35 | 0.35 | 0.35 | 0.35 |
| Purified water | QS | QS | QS | QS |
| Emulsion stability | Stable | Stable | Unstable | Unstable |

Table 2 above shows that stable oil-in-water emulsions were obtained when the HA concentration was 0.2 w/w % or less.

### Example 3

### Incorporation of HA to Form a Stable Emulsion System When the HA Concentration is Low

TABLE 3
Emulsion formulations for Example 3.

| Ingredients | % w/w | % w/w | % w/w | % w/w | % w/w |
|---|---|---|---|---|---|
| Sodium Hyaluronate | 0.05 | 0.2 | 0.3 | 0.5 | 0.7 |
| Castor oil | 0.313 | 0.313 | 0.313 | 0.313 | 0.313 |
| Lumulse GRH-40 | 0.25 | 0.25 | 0.25 | 0.25 | 0.25 |
| PHMB (ppm) | 1 ppm | 1 ppm | 1 ppm | 1 ppm | 1 ppm |
| Dibasic sodium | 0.12 | 0.12 | 0.12 | 0.12 | 0.12 |
| phosphate (7H₂O) | | | | | |
| Monobasic sodium | 0.01 | 0.01 | 0.01 | 0.01 | 0.01 |
| phosphate (H₂O) | | | | | |
| Edetate disodium | 0.01 | 0.01 | 0.01 | 0.01 | 0.01 |
| Taurine | 0.05 | 0.05 | 0.05 | 0.05 | 0.05 |
| Potassium chloride | 0.14 | 0.14 | 0.14 | 0.14 | 0.14 |
| Sodium chloride | 0.75 | 0.75 | 0.75 | 0.75 | 0.75 |
| Purified water | QS | QS | QS | QS | QS |
| Emulsion stability | Stable | Stable | Un- | Un- | Un- |
| | | | stable | stable | stable |

Table 3 shows that stable oil-in-water emulsions were obtained when the HA concentration is 0.2 w/w % or less, even when the emulsion concentration is lowered to one fourth of the concentration of Example 2 (Table 2).

### Example 4

Example 4 illustrates that when the HA concentration was maintained constant at 0.2% w/w, but the emulsion concentration was lowered further to ⅛× concentration, the emulsion/HA compositions became unstable.

TABLE 4
Emulsion formulations for Example 4.

| | 1/8X | 1/4X | 1X |
|---|---|---|---|
| Ingredients | % w/w | % w/w | % w/w |
| Sodium Hyaluronate | 0.2 | 0.2 | 0.2 |
| Castor oil | 0.156 | 0.313 | 1.25 |
| Lumulse GRH-40 | 0.125 | 0.25 | 1 |
| Sodium chlorite | | | 65 ppm |
| WSCP | | | 3 ppm |
| Boric Acid | | | 0.6 |
| Sodium borate decahydrate | | | 0.035 |
| Calcium chloride dihydrate | | | 0.006 |
| Magnesium chloride hexahydrate | | | 0.006 |
| PHMB (ppm) | 1 ppm | 1 ppm | |
| Dibasic sodium phosphate (7H₂O) | 0.12 | 0.12 | |
| Monobasic sodium phosphate (H₂O) | 0.01 | 0.01 | |
| Edetate disodium | 0.01 | 0.01 | |
| Taurine | 0.05 | 0.05 | |
| Potassium chloride | 0.14 | 0.14 | 0.14 |
| Sodium chloride | 0.75 | 0.75 | 0.35 |
| Purified water | QS | QS | QS |
| Emulsion stability | Unstable | Stable | Stable |

The above examples illustrate that when the HA concentration is too high or when the emulsion concentration is not sufficient, the HA/Emulsion combination is unstable. However, stable HA/Emulsion compositions were obtained at HA concentrations of at least 0.2% w/w and emulsion concentrations which are equal to or greater than ¼×. While these examples are shown for HA, stable formulations for other water-soluble polymer demulcents may be determined similarly.

### Example 5

### Effect of Surfactant on Quaternary-Based Antimicrobial Activity

FDA/ISO specified test organisms are listed below:
Serratia marcescens, ATCC 13880
Staphylococcus aureus, ATCC 6538
Pseudomonas aeruginosa, ATCC 9027
Candida albicans, ATCC 10231
Fusarium solani, ATCC 36031

(FDA Premarket Notification (510 k) Guidance Document for Contact Lens Care Products, Appendix B, Apr. 1, 1997 and ISO/FDIS 14729: Ophthalmic optics-Contact lens care products-Microbiological requirements and test methods for products and regimens for hygienic management of contact lenses, January 2001). Contact lens disinfectants are also known as contact lens multi-purpose solutions, when they are used for rinsing, cleaning, disinfection, storage and rewetting contact lenses.

FDA and ISO guidelines specify two disinfection efficacy standards, defined in Table 5 below. Disinfectants are directly challenged with Pseudomonas aeruginosa, Staphylococcus aureus, Serratia marcescens, Candida albicans, and Fusarium solani. The primary criteria for passing state that a minimum 99.9% (3.0 logs) reduction is required for each of the three bacterial types within the minimum recommended soaking period. Mold and Yeast must meet a minimum 90.0% (1.0 log) reduction within the minimum recommended soaking period with no increase (stasis) at not less than four times the minimum recommended soaking period within an experimental error of ±0.5 logs. If the primary criteria is met, the composition may be labeled as a disinfectant.

If the primary criteria is not met the secondary criteria states that the sum of the averages must be a minimum of 5.0 log units reduction for the three species of bacteria within the recommended soaking period with a minimum average of 1.0 log unit reduction for any single bacteria. Stasis for the yeast and mold shall be observed for the recommended soaking period within an experimental error of ±0.5 logs. The composition may be labeled as part of a disinfectant regiment if it passes the second criteria.

TABLE 5
Disinfection efficacy standards.

| | |
|---|---|
| Organism | Average log reduction at labeled soak time |

Stand Alone Disinfectant (Primary) Criteria:

| | |
|---|---|
| S. marcescens | 3.0 logs |
| S. aureus | 3.0 logs |
| P. aeruginosa | 3.0 logs |
| C. albicans | 1.0 log |
| F. solani | 1.0 log |

Regimen-Dependent Disinfectant (Secondary) Criteria:

| | |
|---|---|
| S. marcescens | Minimum of 1.0 log per bacterium, |
| S. aureus | sum of all three bacteria log-drops |
| P. aeruginosa | must be greater than or equal to 5.0 log |
| C. albicans | Stasis |
| F. solani | Stasis |

Antimicrobial activity provided by quaternary-based antimicrobials is frequently lost in the presence of a large amount of surfactant containing alkyl chains, such as POE(40) Hydrogenated Castor Oil. In fact, Tween 80 is routinely used as a quaternary ammonium neutralizer in antimicrobial activity testing. The surfactant forms micelles, which strongly adsorb the antimicrobial, thereby reducing the activity. Table 6 below shows that the alkyl groups in the emulsion can also adsorb the quaternary ammonium molecules thereby inactivating antimicrobial activity.

TABLE 6
Effect of emulsion on log drop

| | | CPC | Alexidine | Alexidine |
|---|---|---|---|---|
| | CPC with | without | with | without |
| Ingredients | emulsion | emulsion | emulsion | emulsion |
| Castor Oil | 0.625 | | 0.625 | |
| Lumulse GRH-40 | 0.500 | | 0.500 | |
| Sodium Hyaluronate | 0.1 | 0.05 | | 0.5 |
| PVP | | | 0.15 | |
| Cetylpyridinium | 5 ppm | 2 ppm | | |
| Chloride | | | | |
| Alexidine | | | 2.5 ppm | 2 ppm |
| Tris HCl | | 0.055 | | 0.055 |
| Tris base | | 0.021 | | 0.021 |
| Pluronic F87 | | 0.05 | | 0.05 |
| Propylene glycol | | 0.5 | | 0.5 |
| Dibasic Sodium | 0.12 | | 0.12 | |
| Phosphate (7H₂O) | | | | |
| Monobasic Sodium | 0.01 | | 0.01 | |
| Phosphate (1H₂O) | | | | |
| Taurine | 0.05 | 0.05 | 0.05 | 0.05 |
| Potassium Chloride | 0.14 | 0.14 | 0.14 | 0.14 |
| Sodium Chloride | 0.75 | 0.59 | 0.75 | 0.59 |
| Edetate Disodium | 0.01 | 0.01 | 0.01 | 0.01 |
| Purified water | QS | QS | QS | QS |

LOG DROP AT 6 HOURS

| | | | | |
|---|---|---|---|---|
| S. marcescens ATCC | 0.81 | 4.1 | 0.41 | 4.9 |
| 13880 | | | | |
| S. aureus ATCC 6538 | 0.15 | 3.98 | 0.35 | 3.3 |
| P. aeruginosa ATCC | 0.31 | 4.56 | 1.52 | 4.6 |
| 9027 | | | | |
| C. albicans ATCC | −0.13 | 2.8 | 0.14 | 1.7 |
| 10231 | | | | |
| F. solani ATCC | 0.15 | 2.44 | 0.25 | 2.9 |
| 36031 | | | | |
| Sum | 1.3 | 17.9 | 2.7 | 17.4 |

As can be seen from Table 6, the log drop in the presence of the surfactant Lumulse GRH-40 is much lower than in the absence of the surfactant. Loss of antimicrobial activity is a problem for ophthalmic compositions. This problem is solved by the ophthalmic compositions according to the invention, where the HLB value of the disinfectant is carefully chosen. These ophthalmic compositions retain antimicrobial activity even in the presence of surfactant as shown below.

### Example 6

### Incorporation of Quaternary Ammonium Antimicrobial into the Emulsion Formulation

The formulation of Table 7 was prepared as described in Example 1. Antimicrobial testing is shown in Table 8.

TABLE 7
WSCP System with Emulsion

| Ingredients | % W/W |
|---|---|
| Castor oil | 0.625 |
| Lumulse GRH-40 | 0.5 |
| Sodium hyaluronate | 0.2 |
| Boric Acid | 0.6 |
| Sodium Borate Decahydrate | 0.03 |
| Calcium chloride dihydrate | 0.006 |
| Magnesium chloride hexahydrate | 0.006 |
| Potassium chloride | 0.14 |
| Sodium chloride | 0.35 |
| Final Volume | 100 |
| pH | 7.5 |
| Sodium chlorite | 65 ppm |
| WSCP | 0.5 ppm |

TABLE 8
Log drops for the Formulation of Table 7
Log Drops

| | | | 7 | 14 | 21 | 28 |
|---|---|---|---|---|---|---|
| Organism | 6 hrs | 24 hrs | days | days | days | days |
| S. aureus ATCC 6538 | 0.5 | 2.4 | 4.8 | 4.8 | 3.9 | 3.9 |
| P. aeruginosa ATCC 9027 | 0.5 | 4.3 | 4.7 | 4.7 | 3.6 | 3.6 |
| E. coli ATCC 8739 | 0.7 | 4.5 | 4.5 | 4.5 | 3.9 | 3.9 |
| C. albicans ATCC 10231 | | | 3.7 | 4.7 | 3.5 | 3.5 |
| A. niger ATCC 16404 | | | 1.0 | 1.0 | 0.4 | 0.5 |

Surprisingly, the antimicrobial activity increases with aging of the HA-containing emulsions and by 7 days, the criteria for primary disinfectant is met. Furthermore, the criteria for preservative efficiency testing as defined below (Table 9) is also met.

TABLE 9
Preservative Efficiency Testing Criteria

| Organism | USP |
|---|---|
| S. aureus ATCC 6538 | 1.0 log at 7 days |
| P. aeruginosa ATCC 9027 | 3.0 logs at 14 days |
| E. coli ATCC 8739 | FDA/ISO: |
| | 3.0 logs at 14 days |
| | 3.0 logs at 28 days |
| C. albicans ATCC 10231 | Stasis |
| A. niger ATCC 16404 | FDA/ISO: |
| | Stasis |

### Example 7

PHMB in HA/Emulsion System

This example shows the HA/Emulsion system with PHMB as the disinfectant. The composition was prepared with the Formulation of Table 10, essentially as described in Example 1. As can be seen by the results of Table 11, at least the secondary regimen-dependent criteria are met by this formulation.

TABLE 10
Formulation for Example 7.

| Ingredients | % W/W |
|---|---|
| Castor oil | 0.625 |
| PEG (40) Hydrogenated Castor Oil | 0.5 |
| Sodium hyaluronate | 0.1 |
| PHMB | 1 ppm |
| Dibasic sodium phosphate (7 H₂O) | 0.12 |
| Monobasic sodium phosphate (1 H₂O) | 0.01 |
| Taurine | 0.05 |
| Potassium chloride | 0.14 |
| Sodium chloride | 0.75 |
| Edetate disodium | 0.01 |
| Purified water | QS |
| Sodium hydroxide (pH adjust) | pH 7.2 |

TABLE 11
Log drop at 6 hours for Formulation of Table 10.

| | Log Drop at 6 |
|---|---|
| Organism | hours |
| S. marcescens ATCC 13880 | 3.77 |
| S. aureus ATCC 6538 | 3.62 |
| P. aeruginosa ATCC 9027 | 4.49 |
| C. albicans ATCC 10231 | 0.33 |
| F. solani ATCC 36031 | 2.76 |

1. A self-emulsifying composition comprising:
oil globules having an average size of less than 1 micron dispersed in an aqueous phase, said globules comprising:
(a) a surfactant component;
(b) a polar oil component, said surfactant component and said oil component selected to self-emulsify when mixed without mechanical homogenization; and
(c) a first therapeutic component comprising a water-soluble polymer.

2. The self-emulsifying composition of claim 1, wherein the surfactant component consists essentially of one or two surfactants.

3. The self-emulsifying composition of claim 1, wherein the water soluble polymer is selected from the group consisting of hyaluronic acid and salts thereof, polyvinylpyrrolidone (PVP), cellulose polymers, dextran 70, gelatin, polyethylene glycols and polyvinyl alcohol.

4. The self-emulsifying composition of claim 3, wherein the cellulose polymer is carboxymethylcellulose or hydroxypropyl methylcellulose.

5. The self-emulsifying composition of claim 3, wherein the polyethylene glycol is PEG 300 or PEG 400.

6. The self-emulsifying composition of claim 1, wherein the oil component comprises castor oil or a natural oil.

7. The self-emulsifying composition of claim 1, further comprising a chlorite preservative component.

8. The self-emulsifying composition of claim 7, wherein the chlorite preservative component is selected from the group consisting of stabilized chlorine dioxide (SCD), metal chlorites, and mixtures thereof.

9. The self-emulsifying composition of claim 1, which further comprises a cationic antimicrobial selected from the group consisting of poly[dimethylimino-w-butene-1,4-diyl]chloride, alpha-[4-tris(2-hydroxyethyl)ammonium]-dichloride (Polyquaternium 1®), poly (oxyethyl(dimethyliminio)ethylene dmethyliminio)ethylene dichloride (WSCP®), polyhexamethylene biguanide (PHMB), polyaminopropyl biguanide (PAPB), benzalkonium halides, salts of alexidine, alexidine-free base, salts of chlorhexidine, hexetidine, alkylamines, alkyl di- and tri-amine, tromethamine(2-amino-2-hydroxymethyl-1,3 propanediol), Octenidine (N,N′-(1,10-Decanediyldi-1-(4H)-pyridinyl-4-ylidenebis-[1-octanamine]dihydrochloride, hexamethylene biguanides and their polymers, cetylpyridinium chloride, cetylpyridinium salts, antimicrobial polypeptides, and mixtures thereof.

10. The self-emulsifying composition of claim 1, wherein the surfactant component has a hydrophobic portion which comprises a first part oriented proximal to the aqueous phase that is larger than a second part of the hydrophobic portion of the surfactant component oriented towards the interior of the oil globule.

11. The self-emulsifying composition of claim 10 wherein the surfactant component consists essentially of one surfactant with the first part of the hydrophobic portion of the surfactant that contains more atoms than the second part of the hydrophobic portion of the surfactant.

12. The self-emulsifying composition of claim 10, wherein the surfactant component consists essentially of two surfactants, a first of said surfactants comprising a first hydrophobic portion and a second of said surfactants comprising a second hydrophobic portion, said first hydrophobic portion having a longer chain length than the second hydrophobic portion.

13. A self-emulsifying composition according to claim 1, further comprising an additional surfactant that does not interfere with self-emulsification.

14. The self-emulsifying composition of claim 1, wherein the surfactant component is selected from the group consisting of (a) a compound having at least one ether formed from at least about 1 to 100 ethylene oxide units and at least one fatty alcohol chain having from at least about 12 to 22 carbon atoms; (b) a compound having at least one ester formed from at least about 1 to 100 ethylene oxide units and at least one fatty acid chain having from at least about 12 to 22 carbon atoms; (c) a compound having at least one ether, ester or amide formed from at least about 1 to 100 ethylene oxide units and at least one vitamin or vitamin derivative; and (d) combinations thereof consisting of no more than two surfactants.

15. The self-emulsifying composition of claim 1, wherein the surfactant component is selected from the group consisting of Lumulse GRH-40 and TPGS.

16. The self-emulsifying composition of claim 1, wherein the oil globules have an average size of less than about 0.25 micron.

17. The self-emulsifying composition of claim 1, wherein the oil globules have an average size of less than about 0.15 micron.

18. The self-emulsifying composition of claim 1, further comprising a cationic antimicrobial component having an HLB value significantly higher than an HLB value of the polar oil component.

19. A therapeutic composition comprising the self-emulsifying composition of claim 1 and a second therapeutic component.

20. The therapeutic composition of claim 19, wherein the second therapeutic component is selected from the group consisting of cyclosporin, prostaglandins, Brimonidine, and Brimonidine salts.

21. A therapeutic composition comprising the self-emulsifying composition of claim 14, further comprising a second therapeutic component.

22. The therapeutic composition of claim 21, wherein the second therapeutic component is selected from the group consisting of cyclosporin, prostaglandins, Brimonidine, and Brimonidine salts.

23. The self-emulsifying composition of claim 1, wherein the self-emulsifying compposition is a multipurpose solution for contact lenses.

24. A method of treating an eye comprising administering the self-emulsifying composition of claim 1 to an individual in need thereof.

25. The method of claim 24, wherein the treatment is for dry eye.

26. The method of claim 24, wherein the individual is a mammal.

27. A method of preparing a self-emulsifying composition comprising:
preparing an oil phase comprising a polar oil and a surfactant component, wherein the polar oil and the surfactant component in the oil phase are in the liquid state;
preparing an aqueous phase at a temperature that permits self-emulsification; wherein the aqueous phase comprises a water soluble polymer; and
mixing the oil phase and the aqueous phase to form an emulsion, without mechanical homogenization.

28. A method of preparing a self-emulsifying composition according to claim 27, further comprising forming a paste between the oil phase and a part of the aqueous phase and mixing the paste with the rest of the aqueous phase to form an emulsion.

29. The method of preparing a self-emulsifying composition according to claim 27, wherein the water soluble polymer is selected from the group consisting of hyaluronic acid and salts thereof, polyvinylpyrrolidone (PVP), cellulose polymers, dextran 70, gelatin, polyethylene glycol and polyvinyl alcohol.

30. The method of claim 29, wherein the cellulose polymer is carboxymethylcellulose or hydroxypropyl methylcellulose.

31. The method of claim 29, wherein the polyethylene glycol is PEG 300 or PEG 400.

32. The method of preparing a self-emulsifying composition according to claim 27, wherein the surfactant component consists essentially of one or two surfactants.
